SEQUENCE LISTING (7 sequences)

SEQ ID NO: 1 - DNA, 21 bp, Artificial
  repeating nucleotide triplet
cttcttcttcttcttcttctt

SEQ ID NO: 2 - DNA, 62 bp, Artificial
  oligonucleotide which contains a repeating nucleotide triplet
gatcgaagaagaagaagaagaagaagaagaagaagaagaagaagaagaagaagaagtatact

SEQ ID NO: 3 - DNA, 62 bp, Artificial
  oligonucleotide which contains a repeating nucleotide triplet
gatcagtatacttcttcttcttcttcttcttcttcttcttcttcttcttcttcttcttcttc

SEQ ID NO: 4 - DNA, 51 bp, Artificial
  repeating nucleotide triplet
gaagaagaagaagaagaagaagaagaagaagaagaagaagaagaagaagaa

SEQ ID NO: 5 - DNA, 19 bp, Artificial
  PCR primer
tttgtgatgctcgtcaggg

SEQ ID NO: 6 - DNA, 24 bp, Artificial
  PCR primer
tttgtgatgctcgtcagggaattc

SEQ ID NO: 7 - DNA, 24 bp, Artificial
  PCR primer
gaattccctgacgagcatcacaaa

(19) United States
(12) Patent Application Publication
(10) Pub. No.: US 2009/0130735 A1
(43) Pub. Date: May 21, 2009

(54) Static Support Bed for Purification, Separation, Detection, Modification and/or Immobilization of Target Entities and Method of Using Thereof

(75) Inventors: Marcos Simon, San Sebastian (ES); Aimara Castelruiz, San Sebastian (ES); Iraida Loinaz, San Sebastian (ES); Jose Adolfo Pomposo, San Sebastian (ES); Valentina Zhukova, San Sebastian (ES)

Correspondence Address: BERENBAUM, WEINSHIENK & EASON, P.C, 370 17TH STREET, SUITE 4800, DENVER, CO, 80202, US

(73) Assignee: DRO BIOSYSTEMS, S. L., San Sebastián (ES)

(21) Appl. No.: 11/996,897

(22) PCT Filed: Jul. 21, 2006
(86) PCT No.: PCT/EP2006/064483

§ 371 Date: Jan. 25, 2008

(30) Foreign Application Priority Data
Jul. 26, 2005 (ES) EP 05106861.7

(51) Int. Cl. C12N 13/00; B01L 11/00; C12M 1/00

(52) U.S. Cl. 435/173.9; 422/99; 435/287.1; 435/287.2

(57) ABSTRACT

The subject matter hereof discloses a static support bed (SSB) for purification, separation, modification, and/or immobilization of target chemical entities or target biological entities present in a fluid. The static support bed hereof may include one or more microwire supports suitable for the attachment of target chemical entities or target biological entities.

The present subject matter relates to a static support bed for purification, separation, modification and/or immobilization of target chemical entities or target biological entities present in a fluid.

### BACKGROUND

Different analytical, biochemical and diagnostic methods involve immobilization of a specific reagent or a biological binding partner of a biological molecule onto high surface area substrates.

On one hand, cells are often cultured in reactors to produce biological and pharmacological products. Such cells can be animal, plant, fungi, or microbial cells. In order to maintain a cell culture, oxygen and other nutrients generally must be supplied to the cells. Cell cultures are usually maintained in reactors by perfusion, wherein a cell culture medium, including oxygen and other nutrients, is directed through the cell culture reactor. Cell-culture reactors, however, can support only small cell loadings per unit of reactor volume. They can only operate within low flow rate or agitation rates. Similarly, biocatalytic reactions are performed in reactors, where an enzyme catalyst is retained on a porous inorganic support.

On the other hand, immobilization has also been used to perform chemical and biological separations. Separation of macromolecules such as proteins has a considerable cost in the manufacture of pharmacological products. Chromatography has been used for decades to perform such type of separations. Chemically modified cellulose or silica are used for the stationary phase in the manufacture of commercially important biomolecules in the food, biopharmaceutical, biotechnology and pharmaceutical industries. Alternative stationary phases can include metals and metal oxides, for example, particulate aluminum oxide. Membrane adsorbents, i.e. membranes with functionalized sites on the surface for chromatography, can also be used.

Many analytical methods involve immobilization of a biological binding partner of a biological molecule on a surface. The surface is exposed to a medium suspected to contain the molecule, and the existence or extent of molecule coupling to the surface-immobilized binding partner is determined.

Likewise, many biotechnological processes for producing pharmaceutical or diagnostic products involve the purification of biomolecules from a variety of sources. Purification of a biomolecule is often initiated via the use of adsorption chromatography on a conventional packed bed of solid support adsorbent. This frequently requires clarification of the crude culture before application onto the chromatography column. Actual processes of production and purification of plasmidic DNA from bacterial lysates, are based on conventional packed bed chromatography. This method is hampered by the physical characteristics of these compounds (e.g. size of plasmids, viscosity of solutions, fragility of plasmids, chemical similarity with other nucleic acids from the microorganism, etc.), setting stringent limitations in terms of operating bed capacity and pressure drop. Furthermore, it may be necessary to eliminate the plasmid fraction that does not contribute to the therapeutic effect due to the fact that the expression of the genes contained in the non-therapeutic portion entails a danger for the receptor of such plasmid, such as risk of unspecific effects, risk of chromosomal integration, inter alia.

Adsorption chromatography methods may be carried out not only on a conventional packed bed (packed bed chromatography; PBC), but also on an expanded bed (expanded bed adsorption; EBA) or a fluidized bed (fluidized bed adsorption; FBA). All of these chromatographic methods contain particles as adsorption support.

Other chromatographic methods used in separation and purification, use fibrous media as stationary phase. Thus, J. Chromatography 1992, 598/2: pp. 169-180 describes, for example, a continuous stationary phase consisting of yarns woven into a fabric rolled and packed into liquid chromatography columns. The yarns described have a characteristic width of 200-400 μm, are made from 10-20 μm fibers of 95% poly(m-phenylene isophthalamide) and 5% poly(p-phenylene terephthalamide).

J. Oleo Science 2002, 51/12: 789-798, describes a liquid chromatography method with polyester and cellulosic filament yarns as the stationary phase to remove oily soils from a fiber substrate with an aqueous surfactant micellar solution.

EP 328256 discloses a glass fiber coated with a porous hydrophilic matrix material which is derivatized to bind a suitable ligand or biological material in a chromatographic process.

Finally, WO 03/00407 relates to aluminum hydroxide fibers highly electropositive and approximately 2 nanometers in diameter. Such fibers can filter bacteria and nano size particulates such as viruses and colloidal particles at high flux through the filter. They can also be used for purification and sterilization of water, biological, medical and pharmaceutical fluids, as a collector/concentrator for detection and assay of microbes and viruses, and also as a substrate for growth of cells.

Amorphous glass coated microwires are known in the art. Due to their magnetic properties at high frequencies they may have been used in miniature electronic components, and for filtering of electromagnetic interference in printed circuits and cables. The microwires have also conducting properties and, therefore, may be used in electromagnetic applications like: miniature coils, miniature cables, high voltage transformers and miniature antennas. Nevertheless, it is unknown whether use of the amorphous glass coated microwires has been proposed as a method for purification, separation, modification and/or immobilization of target substrates contained in a fluid.

J. Magnetism and Magnetic Materials 2002, 249: 357-367 describes the use of nanoporous membranes partially filled with magnetic hollow wires to separate magnetic beads present in a fluid. The magnetic beads, previously loaded with specific biological entities, are separated by passing the fluid containing the magnetic beads through the membrane while applying an external magnetic field in order to magnetize the ferromagnetic cylinders, and therefore the biological entities bound to the magnetic beads are trapped on the walls of the capillaries while the unbound units may be passed through.

J. Magnetism and Magnetic Materials 2005, 293: 671-676 describes the sensitivity of glass covered amorphous microwires to the Giant Magnetoimpedance effect (GMI) for the detection of magnetic microparticles settled on and near its surface when a magnetic field is applied. The microwire is covered with a polymer containing specific bioreceptors for the target biomolecules present in the surface of the magnetic microparticles which are subsequently detected.

WO 2005/101464 relates to metallic glass coated microwires wherein the biochemical reagents and enzymes of the PCR reaction are encapsulated or loaded into nano- or micropores etched on the glass surface that is either a part of the glass-coated microwire or is deposited thereon by dipping, spraying, or some other method.

### SUMMARY

Disclosed herein is a method for purification, separation, modification and/or immobilization of target chemical entities or target biological entities present in a fluid and in some implementations, avoiding or minimizing one or more of the inconveniences above mentioned for other methods.

This method may be carried out using a static support bed containing one or more microwire supports secured by their ends as a stationary phase, said microwire supports being suitable for the attachment of target chemical entities or target biological entities.

Accordingly, a first aspect hereof relates to a static support bed (SSB) for purification, separation, modification, and/or immobilization of target chemical entities or target biological entities present in a fluid, where the static support bed has one or more microwire supports secured by at least one of their ends, these microwire supports having a multilayer structure segregated into a central core and one or more coating layers, and being suitable for the attachment of target chemical entities or target biological entities, with a proviso that if a magnetic field is acting upon said static support bed then the magnetic field is not used to separate and/or detect magnetically susceptible particles present in the fluid through the magnetic interaction established between the microwire supports and said magnetically susceptible particles.

Microwire supports are also subject-matter hereof. Thus, a second aspect relates to the microwire supports which may be integrated in the static support bed hereof, having the features mentioned above, the microwire supports having a multilayer structure segregated into a central core and one or more coating layers, wherein the surface of the microwire may be modified by:

a) attachment of ligands; or

b) by coating it with a functional coating, with a proviso that the functional coating is not a polymeric coating.

A third aspect relates to a method for purification, separation, modification, and/or immobilization of target chemical entities or target biological entities present in a fluid, using a static support bed as defined above, wherein the method includes: a) loading a sample fluid containing the target chemical entities or target biological entities into the inner volume of a channel containing the static support bed; b) attaching the target chemical entities or target biological entities on the microwire supports of the static support bed; c) optionally, carrying out a chemical or biological modification on the target entity, such as a biocatalytical modification of the target molecule; d) optionally, washing the channel and discharging undesired components and impurities of the sample fluid; and e) eluting the resulting chemical entities or the resulting biological entities, with a proviso that if a magnetic field is acting upon said static support bed then the magnetic field is not used to separate and/or detect magnetically susceptible particles present in the fluid through the magnetic interaction established between the microwire supports and said magnetically susceptible particles.

This method may be used for the separation, purification and/or modification of biomolecules such as proteins, glycoproteins, nucleic acids, such as RNA, DNA, cDNA, oligonucleotides and plasmids, peptides, hormones, antigen, antibodies, lipids and complexes including one or more of these molecules.

### DEFINITIONS

In the following the term “biological entity” may include components of biological origin. It may include animal, plant, fungi, or microbial cells, tissue cultures, antibodies, antibiotics, antigens, plasmids, oligonucleotides, peptides, hormones, coenzymes, enzymes, proteins, either naturally or recombinantly produced, glycosylated or not, cellular components, nucleic acids, viruses, carbohydrates, body fluids, blood components, microorganisms, and derivatives thereof, or parts thereof as well as any other biological molecule of interest.

As used herein, the term “chemical entity” may include any organic or inorganic compound, including drugs.

As used herein, the term “purification” may refer to the process of separating a substance of interest from foreign or contaminating elements in a sample by removing impurities.

As used herein, the term “separation” may refer to a process that transforms a mixture of substances into two or more compositionally-distinct products.

As used herein, the term “modification” may refer to an alteration in the structure of a molecule by chemical or biological means.

As used herein, the term “immobilization” may refer to the act of attaching by covalent or non-covalent forces a chemical compound or a biomolecule. Immobilization of cells, to produce vaccines, proteins, eukaryotic genes, tissue grafts, proteins from recombinant DNA, etc. one use of the microwires hereof.

As used herein, the term “maximum cross-sectional dimension” of any given object may refer to the maximum distance found between any two given points contained within the largest perimeter defined by the intersection of the object and a plane perpendicular to the longest dimension of the object.

The term “microwire”, as used herein, may refer to a solid, i.e. not hollow, thin element, which may be of circular or non-circular cross-section, and which may have a maximum cross-sectional dimension smaller than about 1000 μm. The terms “microwire” and “microwire support” are used interchangeably in this document.

As used herein the term “static support bed” may refer to a matrix composed of one or more microwire supports, which often, if more than one, may be grouped together in a recurring pattern and immobilized by either end.

The term “bundle of microwire supports”, as used herein, may refer to a plurality of microwire supports, i.e. more than one microwire support grouped together in a recurring pattern.

### DETAILED DESCRIPTION

An important issue in present day industrial processes is scalability limitation due to the technology applied. This limitation may result in a successful process on a small laboratory scale failing to yield the expected results when applied at large industrial scale.

Therefore, the dimensions of static support bed devices, according to the present subject matter, may cover the range from about 0.5 cm of maximum cross-sectional dimension and about 0.5 cm in length to about 1.5 m of maximum cross-sectional dimension and about 10 m in length. However, due to the high capacity of static support bed technology, the use of very large static support bed devices is very rare therefore, dimensions of static support bed devices for industrial processes may cover the range from about 0.5 cm of maximum cross-sectional dimension and about 5 cm in length to about 50 cm of maximum cross-sectional dimension to about 1.5 m in length.

Static support bed technology may provide a large available surface area combined with high porosity within the boundaries of a static support bed device. This results from the filamentous shape of the microwire supports employed in this development. Given a particular porosity of the static support bed device, a larger available surface area may be provided within the static support bed device when thinner microwire supports are employed. However, thicker microwire supports may be more resistant to fracture, and for this reason large devices or harsh process conditions may require the use of thicker microwire supports.

The length of the microwire supports is not particularly restricted to any specific range, insofar as generally it may be equal to or larger than the length of the column or reactor. Nevertheless, the above mentioned considerations illustrate the convenience of using the microwire supports having a maximum cross-sectional dimension in the range of about 1 μm to about 1000 μm and, to maintain their filamentous shape, a length to maximum cross-sectional dimension ratio larger than about 5. More preferably, their maximum cross-sectional dimension is in the range of about 1 μm to about 100 μm and the length to maximum cross-sectional dimension ratio is larger than about 50. Even more preferably, the length to maximum cross-sectional dimension ratio is larger than about 500. Most preferably, the length to maximum cross-sectional dimension ratio is larger than about 1000.

Static support bed technology, as described herein may be applied to the processing of large fluid volumes, fast flowing fluids, viscous fluids and fluids with solids in suspension. In any of these four instances, application of existing technologies often result either in low productivity or in limiting backpressure.

Backpressure may arise when the processing device interposed in the flow of the fluid that is being processed exerts resistance to the flow. This resistance may be the consequence of the large viscosity or large flow speed applied compared to the porosity at any given cross-section of the device. The porosity of the device may be affected by the design of the device, the size and geometry of the supports contained within the device and also by the filtering effect on solids in suspension that accumulate within the device and reduce the effective porosity of the device.

In static support bed technology, microwire supports may be secured at either end to provide a static support bed where the spatial distribution of microwire supports may remain stable independently of the nature of the fluid applied or the velocity of the flow applied. The securing of the ends may thus result in adjacent microwire supports forming a particular angle. In order to avoid backpressure and filtering effect on solids in suspension, a convenient arrangement of the microwire supports within the static support bed device is when adjacent microwire supports form an angle of zero degrees with each other. However, solids of a dimension larger than the distance between adjacent microwire supports may pass relatively unhindered through the bed by pushing away adjacent microwire supports and distorting temporarily the spatial distribution of adjacent microwire supports that may result in adjacent microwires forming an angle of up to ten degrees. Moreover, design and construction limitations may require increasing the angle between adjacent microwire supports up to forty-five degrees.

Therefore, according to one implementation of the present developments, any given individual microwire support forms an angle between about 0° and about 45° with any other neighboring microwire support. Any given individual microwire support may form an angle between about 0° and about 10° with any other neighboring microwire support.

In one implementation, the microwires may be placed in the column or reactor in such a manner that they are extended from one end to the other end of the column or reactor, immobilized by their ends, and the feeding flow may run through one end of the column or reactor to the other end. In another implementation, the microwire supports may be placed in such a way that the feeding flow makes an angle between about 0° and about 45° with the microwire supports. Nevertheless, other dispositions may also and/or alternatively be allowed.

A total coverage of the inner volume of the column or reactor with microwire supports may call for a uniform distribution throughout the column or reactor of the microwire supports, as such or grouped in bundles. Such a uniform distribution may be achieved by immobilizing the microwire supports or bundles by their ends, on either end of the column or reactor in such a way that the immobilized ends may form a grid, zigzag or parallel line pattern on either end of the column or reactor.

Accordingly, a static support bed, also referred to as an SSB may be placed within the column or reactor in an optimised distribution pattern to achieve the desired values of scale and uniform bed porosity. These values may be kept constant throughout the life span of an SSB device.

In one implementation of the subject matter hereof, the microwire supports of a static support bed may have a central core and coating layers made of different materials. Therefore, the microwire supports according to this implementation do not present a hollow structure, unlike the hollow-fiber like wires. The materials of the central core and the coating layers may be glass, metallic, ceramic, polymeric or plastic material. In another implementation, the central core of the microwire supports may be made of metal, and at least one coating layer may be made of glass. The metallic core of the microwire supports may have an amorphous and/or crystalline microstructure.

In another implementation, the core of the microwire supports may be made of a metal, metallic alloys or combinations of at least one metal and a metal alloy. Metals used as such or in alloys may be copper, gold, silver, platinum, cobalt, nickel, iron, silicon, germanium, boron, carbon, phosphorus, chromium, tungsten, molybdenum, indium, gallium, lead, hafnium or zirconium. The core of the microwire support may be of an alloy containing cobalt, iron, nickel, chromium, boron, silicon and molybdenum.

Examples of composition of cores in the present microwire supports are those included in Table 1.

TABLE 1

| | Co % | Fe % | Ni % | Cr % | B % | Si % | Mo % |
|---|---|---|---|---|---|---|---|
| 1 | 68.7 | 4 | 1 | 0 | 13 | 11 | 0 |
| 2 | 50.7 | 3.98 | 0 | 23.65 | 11.96 | 9.71 | 0 |
| 3 | 60.51 | 3.99 | 0 | 12.13 | 13.53 | 9.84 | 0 |
| 4 | 59.85 | 3.94 | 0 | 12 | 13.38 | 10.83 | 0 |
| 5 | 58.34 | 3.84 | 0 | 11.7 | 13.06 | 13.06 | 0 |
| 6 | 58.14 | 4.17 | 0 | 11.66 | 13.02 | 13.01 | 0 |
| 7 | 58.9 | 4.19 | 0 | 12.42 | 13.13 | 11.36 | 0 |
| 8 | 58.64 | 4.67 | 0 | 12.36 | 13.05 | 11.28 | 0 |
| 9 | 57.33 | 4.7 | 0 | 13.14 | 13.02 | 11.19 | 0.62 |
| 10 | 56.51 | 4.84 | 0 | 13.08 | 14.16 | 11.41 | 0 |
| 11 | 58.04 | 4.62 | 0 | 12.92 | 12.8 | 11.01 | 0.61 |
| 12 | 58.25 | 4.49 | 0 | 12.52 | 13.47 | 10.68 | 0.59 |
| 13 | 57.96 | 4.73 | 0 | 12 | 13.2 | 11.11 | 1 |

The vitreous coating composition may include metal oxides such as SiO₂, Al₂O₃, B₂O₃, Na₂O and K₂O, among others.

In an implementation of the present subject matter, the surface of the microwire supports may be modified by attachment of ligands or by coating it with a functional coating, therefore the purification, separation, modification, and/or immobilization may occur through the attachment of the target chemical entity or target biological entity to the functional coating or to the ligand present in the surface of the microwire supports.

In another implementation of the subject matter hereof, the surface of the microwire supports may be modified by coating the surface with a proteic, gelatin, or collagen coating. Therefore, in this case, the surface of the microwire support may be modified by a functional coating. The term “functional coating”, as used herein, refers to a coating which may interact by covalent or non-covalent coupling with the target entity.

In another implementation of the subject matter hereof, the surface of the microwire supports may be modified by attachment of a ligand to the surface of the microwire support, directly or through a linker. Ligands may be cells, biological tissues, antibodies, antibiotics, antigens, nucleic acids, peptides, hormones, coenzymes, biological catalysts, chemical catalysts, chemical reactants, lipids, sugars, amino acids, proteins, nucleotides, a compound containing a functional group such as diethylaminoethyl, quaternary aminoethyl, quaternary ammonium, carboxymethyl, sulphopropyl, methyl sulphonate, butyl, octal, and phenyl, or mixtures thereof, particularly, cells, biological tissues, antibodies, antibiotics, antigens, nucleic acids, peptides, hormones, coenzymes, biological catalysts, chemical catalysts, chemical reactants, lipids, sugars, aminoacids, proteins, nucleotides, or mixtures thereof.

Linkers may be polymeric coating, proteic coating, gelatin coating, collagen coating, cells, antibodies, antigens, nucleic acids, peptides, coenzymes, lipids, sugars, aminoacids, proteins, nucleotides, cyanuric chloride, quinine, p-mercurybenzoate, phenyl boronic acid, and a compound containing a functional group of aldehyde, aromatic amine, nitrene, maleimide, carboxylic acid, isocyanate, diethylaminoethyl, quaternary aminoethyl, quaternary ammonium, carboxymethyl, sulphopropyl, methyl sulphonate, butyl, octal, and phenyl, or mixtures thereof.

The glass-coated microwires may be prepared by any suitable method known in the art, such as Taylor-Ulitovski method (Fizika Metallov I Metallovedeneie 1989, 67: 73). Different metallic compositions of the core may be used, as well as different compositions of the coating glass may be used.

A functionalized glass-coated microwire support as defined above may be prepared by a process including the following steps: (i) providing a glass-coated microwire support; (ii) oxidizing its surface; (iii) activating the surface of the resulting oxidized microwire; and (iv) functionalizing with an appropriate ligand through covalent or non-covalent coupling of the ligand to the linker attached in step (iii).

The oxidation step (ii) may involve a treatment with H₂O₂/NH₃ aq. (1:4) followed by a treatment with H₂SO₄ conc. Other oxidizing conditions may also be used (cf, J. Am. Chem. Soc; 2003, 125, 12096; Langmuir, 2004, 20, 7753; Anal. Chem.; 1993, 65, 1635; J. Am. Chem. Soc; 1996, 118, 9033).

The activation step (iii) may include attachment of a suitable linker to the surface of the microwire which contains suitable functional groups for covalent or non-covalent (electrostatic, hydrophilic, hydrophobic or affinity interaction) coupling to the ligand. The activating step (iii) of the microwire supports may be performed in a single step or through several reaction steps. For example, the activating step (iii) may be carried out by a process of the following steps: (iii-1) reacting the oxidized microwire to a silane compound; and (iii-2) reacting the resulting microwire product of step (iii-1) to a compound containing a maleimide, carboxylic or isocyanate group. Silane compounds may be 3-aminopropyletoxisilane, 7-oct-1-eniltriclorosilane and 3-isocianotepropyltrietoxisilane.

The functionalization step (iv) may be carried out by coupling the linker attached to the surface of the microwire supports to the ligand through electrostatic interactions, hydrophilic interactions, hydrophobic interactions, affinity interactions or covalent bonds. That coupling may be achieved using any of the following combinations:

a) covalent coupling of ligands:
a.1) an amine function on the ligand linked via imine bond to aldehyde function on the surface.
a.2) an amine function on the ligand bound via nucleophilic substitution of the surface functionalized with cyanuric chloride.
a.3) an amine function on the ligand bound via Michael additions to quinone functions on the surface.
a.4) a tyrosine or histidine residue on the ligand bound through an azo group to aromatic amines linked to the surface.
a.5) an amine residue on the ligand bound to a nitrene function on the surface generated through photochemical activation of phenylazide groups.
a.6) a thiol function on the ligand bound to p-mercurybenzoate, iodoacetamide or maleimide groups on the surface via siloxane bridging, disulfide bonds or Michael addition.
a.7) a cis-diol site (present on the sugars of glycoproteins) on the ligand can be bound to phenyl boronic acid groups on the surface.
a.8) carboxylic or isocyanate groups on the surface bond to amine groups on the ligand.
b) non-covalent coupling of ligands:
b.1) electrostatic interaction, as for example the interaction through charged thiols between a self-assembled monolayer of octadecylthiol and dodecylthiol on the surface and fumarate reductase
b.2) hydrophilic or hydrophobic interactions, as for example an ATPase embedded in a liposome bound to the surface through the interaction of the liposome to a layer of dimyristoylphosphatidylethanolamine on the surface.
b.3) affinity interactions, as for example: antibody-labelled ligands bound to antigen-coated surfaces, biotin-labelled ligands bound to avidin or streptavidin coated surfaces, glycoproteins bound to lectin coated surfaces, alpha-D-mannopyranose containing ligand bound to concanavalin A coated surfaces, choline-binding domain on the ligand bound to choline coated surfaces, FAD-dependent enzyme bound to FAD (flavin adenine dinucleotide) coated surfaces, and cofactor dependent enzymes bound to cofactor analogue coated surfaces.

The static support bed adsorption method described herein, may be used in different applications. Thus, it may be used in a method:

(i) as a biocatalytical reactor by immobilizing enzymes on the surface of the microwire supports;

(ii) to modify target chemical or biological molecules by use of a catalyst, whether biocatalyst or not, bound to the surface of the microwire support;

(iii) to separate target chemical or biological molecules from the fluid in which they are contained, through the interaction of said target chemical or biological molecules with interacting entities bound to the surface of the microwire support;

(iv) to simultaneously separate and modify target chemical or biological molecules contained in a fluid through the action of a catalyst on said target chemical or biological molecules while bound to an interacting entity, being both the catalyst and the interacting entity or only one of them bound to the surface of the microwire support;

(v) to immobilize target chemical or biological molecules which further interact with target chemical or biological molecules contained in the fluid by any of the means described above;

(vi) to modify the composition of a fluid through the activity of cells on the components of said fluid, being said cells bound to the surface of the microwire support;

(vii) to multiply the number of dividing cells by having said cells divide on the surface of the microwire support;

(viii) to modify the composition of a fluid by exchanging target chemical or biological molecules contained in said fluid with target chemical or biological molecules bound to the surface of the microwire support;

(ix) to develop chemical reactions involving one or more than one step through the action of one or more than one agent on molecular entities bound to the surface of the microwire support, i.e. as solid phase synthesis support;

(x) to modify the physical properties of a fluid through the activity of different entities immobilized on the surface of the microwire support or through the action of physical forces conveyed to the fluid through the microwire support;

(xi) to purify plasmid DNA through the interaction of said plasmid DNA with the surface of microwire supports functionalized with oligonucleotides which may be complementary to a target sequence inserted into the plasmid DNA;

(xii) for biocatalytical modification of plasmids through functionalization of the surface of the microwire supports with suitable oligonucleotides which are complementary to a target sequence inserted into the plasmid, and a restriction enzyme and a ligase enzyme;

(xiii) for immobilization and cultivation of cells on the surface of the microwire supports. These microwires with immobilized cells on their surface may be used as biofermentors for cell growth;

(xiv) for solid-phase PCR by immobilization of suitable primers for that method;

(xv) to decontaminate of fluids by immobilizing contaminating agents on the surface of the microwire supports; and/or

(xvi) for any of the above mentioned applications when dealing with viscous fluids with high concentration of solids in suspension and/or with high-speed flow.

In a particular implementation of the method, a magnetic field or electric current may be applied through the static support bed, to aid achieving proper agitation of the microwire support and elution of bound substances on the surface of the microwire support or to adjust the temperature of the microwire support. An electric current may be applied through the microwires, and when so applied, the temperature of the static support bed may be regulated.

Furthermore, a magnetic field may be applied to the static support bed, and when so applied, the method of the present subject matter may be used to separate magnetically susceptible particles from the fluid in which they are contained through the magnetic interaction established between the microwire support and the magnetically susceptible particles.

Compared with other chromatographic methods known in the art, the static support bed adsorption method described by the present subject matter has features such as those shown in Table 2.

TABLE 2

| | | fluidized | expanded | static |
|---|---|---|---|---|
| | packed bed | bed | bed | support |
| | chromatography | adsorption | adsorption | bed |
| Resolution | very high | very low | medium | high |
| Max. | very low | low | low | very high |
| viscosity of | | | | |
| fluid | | | | |
| Max. solid | very low | very high | high | very high |
| content | | | | |
| Max. flow | very low | medium | medium | very high |
| velocity | | | | |
| Scalability | bad | very good | good | very good |
| Geometry | particles | particles | particles | microwires |
| Height of | constant | variable | variable | constant |
| bed as | | | | |
| function of | | | | |
| flow velocity | | | | |
| Porosity | very low | very high | very high | very high |
| | (constant with | (variable | (variable | (constant |
| | flow) | with | with | with flow) |
| | | flow) | flow) | |

Therefore, a positive feature of static support bed, aka SSB, technology may be its scalability. Microwire supports may be produced at the desired length and assembled to fill the desired column diameter. Besides standardised sizes, the devices may be customised to meet particular requirements. Furthermore, SSB technology may also present the following features:
a generally reduced number of downstream processing steps;
operational parameters generally independent of flow velocity;
a generally larger specific surface area than particulate process supports.

Static support bed, SSB, may provide a seamless technology that may facilitate production through improved processability, and may include:

better resolution than competing technologies;

suitability for highly viscous liquors and solids content;

decreased leakage of support particles to the product.

Therefore, according to a method hereof for purification, separation, modification, and/or immobilization of target chemical entities or target biological entities present in a fluid as described herein, the fluid containing the target entities may be passed through an SSB (static support bed) device, then the target entities will specifically bind to the functional coating of the modified surface of the microwire supports, and/or interact with the ligands present in the surface of the microwire supports, while impurities and the fluid may be pass by unhindered. If necessary, a chemical or biological modification can be carried out on the target entity. Optionally, additional steps of washing the channel and discharging undesired components and impurities of the sample fluid can be carried out and finally the resulting chemical entities or the resulting biological entities may be eluted or desorpted and recovered.

In a further implementation, microwire supports provide an excellent surface for growth of adherent cells. Disposable SSB devices may be designed to provide a sterile growth surface and continuous supply of fresh culture media. The system may allow for continuous extracellular protein production and for cell production following a harvesting step.

In another implementation, the use of SSB as solid support for solid phase synthesis may provide an increase of the productivity of solid phase synthesis by providing a higher specific area and faster flow conditions with improved processability.

Throughout the description and claims the word “comprise” and variations of the word, such as “comprising”, is not intended to exclude other technical features, additives, components, or steps. Additional objects, advantages and features of the subject matter hereof will become apparent to those skilled in the art upon examination of the description or may be learned by practice hereof. The following examples are provided by way of illustration, and are not intended to set the limits of the present subject matter.

### EXAMPLES

### Example 1

### Example of Microwire Support Production

The production of a continuous microwire support with an external diameter of 24.4 micrometers is described:

A glass tube with an external diameter of 7 to 10 mm and wall thickness of 1.0 to 1.4 mm filled with a metallic alloy consisting of 69% cobalt, 4% iron, 1% nickel, 13% boron and 11% silicon was fed at a feeding speed between 0.9 and 1.5 mm min−1 to the induction oven of a microwire production machine. The oven temperature was set between 1,260 and 1,330° C. The resulting metal-filled, microwire support was cooled with running water and wound at a winding speed between 150 and 250 m min⁻¹ to form a spool that was stored at room temperature until use.

The thickness of the glass layer of the microwire support and the total diameter of the microwire support can be modified by adjusting the temperature of the induction oven, the winding speed and the feeding speed.

### Example 2

### Production of EcoR I-Activated Microwire Support

### C—S Bond

The microwire support was treated with a mixture of seven volumes of sulphuric acid and three volumes of 30% hydrogen peroxide for thirty minutes at room temperature. The support was then thoroughly rinsed in running water, then in ethanol and then in chloroform. Finally the support was dried in a nitrogen stream. Then the microwire support was treated with 2% (3-aminopropyl)triethoxisylane in water under nitrogen atmosphere at room temperature. Then the support was rinsed in dichloromethane and exposed to a nitrogen stream. Following an ethanol wash, the microwire support was treated with 2 mM 4-maleimidobutyric acid N-hydroxysuccinimide ester in ethanol for 16 h and rinsed in ethanol. The microwire support was then treated with 600 units of EcoR I enzyme per meter of microwire support in TE buffer, pH 8.0 (0.1 M tris(hydroxymethyl)aminomethane and 1 mM ethylendiaminetetraacetic acid in water; pH adjusted to 8.0 with hydrochloric acid) for 16 h and washed in TE buffer.

### Example 3

### Application of EcoR I-Activated Microwire Support to the Treatment of a EcoR I Sensitive Plasmid

2.5 ug mL⁻¹ of pCMS-EGFP plasmid (BD Biosciences, Catalogue number 6101-1) containing a unique target site for EcoR I enzyme was exposed to the activity of EcoR I-activated micro-wire support for 4 hours in an aqueous solution containing 50 mM NaCl, 100 mM tris(hydroxymethyl)aminomethane, 10 mM MgCl₂ and 0.025% Triton X-100 at pH 7.5 and 37° C. The activity of the EcoR I-activated micro-wire support on the plasmid molecules was analyzed by agarose gel electrophoresis. Non-activated microwire support was used as negative control.

### Electrophoretic Analysis of the Activity of EcoR I-Activated Micro-Wire Support on the Plasmid Molecules

600 μL samples of the supernatant obtained following treatment with EcoR I-activated micro-wire support were precipitated in 70% ethanol, solubilised in water and electrophoresed for 40 minutes at 10.5 Vcm⁻¹ in a 0.8% agarose gel in TAE (0.04 M tris(hydroxymethyl)aminomethane; 0.001 M ethylenediamine tetraacetic acid, pH adjusted to 8.5 with glacial acetic acid), using TAE as running buffer. The gel was stained in 0.5 μg mL⁻¹ Ethidium bromide in TAE for 20 minutes and observed under ultraviolet light. Only EcoR I-activated micro-wire support had any effect on the plasmid molecules.

### Example 4

### Production of Avidin-Activated Microwire Support (Amida Bond and Urea Bond)

The microwire support was incubated for 20 minutes in a solution consisting of 1 volume of 33% hydrogen peroxide and 4 volumes of concentrated ammonia. The microwire support was then washed three times in water and treated twice with concentrated sulphuric acid for 30 minutes. The microwire support was then thoroughly rinsed in water and sonicated for 10 minutes in water, rinsed in ethanol and dried in a nitrogen stream. Then two different procedures, Procedure 1 or Procedure 2, were followed to obtain the avidin-activated microwire support.

### Procedure 1 (Amida Bond):

The microwire support was then incubated in dichloromethane containing 2% of 7-oct-1-enyltrichlorosil for 16 hours at room temperature under nitrogen atmosphere and then rinsed first in dichloromethane, second in methanol and finally in water. The resulting microwire support was incubated in an aqueous solution of 0.5 mM KMnO₄, 14.7 mM NalO₄ and 3 mM K₂CO₃ for 24 hours and then washed in water and treated with an aqueous solution of 0.05 M N-Hydroxysuccinimide and 0.2 M N-(3-Dimethylaminopropyl)-N′-ethylcarbodiimide hydrochlorade for 7 minutes. The microwire support was then rinsed in phosphate buffer saline (PBS) pH 7.0 and incubated for 16 hours in PBS containing 0.2 mg mL⁻¹ avidin. The avidin-activated microwire support was washed in water, then in 20% ethanol, dried in a nitrogen stream and kept at room temperature until use.

### Procedure 2 (Urea Bond):

The microwire support was then treated with 2% 3-(Isocyanatopropyl)-triethoxysilane in Dichloromethane at room temperature for 16 hours under nitrogen atmosphere. The resulting microwire support was incubated in Dimethylformamide containing 0.2 mg mL⁻¹ avidin for 1 hour at room temperature and thoroughly washed in water.

### Example 5

### Application of Avidin-Activated Microwire Support to the Immobilization of Biotin-Bound Substrates

The ability of avidin-activated microwire support to bind biotin was analyzed in the following way. Avidin-activated microwire support was incubated with fluorescein-biotin conjugate in Phosphate buffered saline (PBS) for 45 minutes at room temperature. Then the support was washed in PBS and the fluorescence emitted by the biotin-bound fluorescein on the surface of the avidin-activated microwire support was observed in a microscope under ultraviolet light. Non-activated microwire support was used as negative control.

### Example 6

### Production of Oligonucleotide-Activated Microwire Support

### Procedure 1 (Amida Bond)

The microwire support was incubated for 20 minutes in a solution consisting of 1 volume of 33% hydrogen peroxide and 4 volumes of concentrated ammonia. The microwire support was then washed three times in water and treated twice with concentrated sulphuric acid for 30 minutes. The microwire support was then thoroughly rinsed in water and sonicated for 10 minutes in water, rinsed in ethanol and dried in a nitrogen stream. The microwire support was then incubated in dichloromethane containing 2% of 7-oct-1-enyltrichlorosil for 16 hours at room temperature under nitrogen atmosphere and then rinsed first in dichloromethane, second in methanol and finally in water. The resulting microwire support was incubated in an aqueous solution of 0.5 mM KMnO₄, 14.7 mM NalO₄ and 3 mM K₂CO₃ for 24 hours and then washed in water and treated with an aqueous solution of 0.05 M N-Hydroxysuccinimide and 0.2 M N-(3-Dimethylaminopropyl)-N′-ethylcarbodiimide hydrochlorade for 7 minutes. The microwire support was then rinsed in phosphate buffer saline (PBS) pH 7.0 and incubated for 16 hours in PBS containing 10 nmol of H₂N-d(CTT)₇ oligonucleotide per square meter of microwire support surface. The oligonucleotide-activated microwire support was washed in water, then in ethanol, dried in a nitrogen stream and kept at room temperature until use.

### Procedure 1 (Avidin-Biotin Bridge):

Alternatively, oligonucleotide-activated microwire support was produced by incubating avidin-activated microwire support in phosphate buffered saline (PBS), pH 7.5 containing 0.2 μM biotin-d(CTT)₇ oligonucleotide for 45 minutes followed by a wash step in PBS.

### Example 7

### Application of Microwire Support as Substrate for Cell Growth

Sterile microwire support was incubated for two hours in a vessel containing culture medium for eukaryotic cell growth. All the process was carried out in sterility conditions. Then, a suspension of a eukaryotic cell line at 1.5×10⁶ cells per mL was poured into the vessel containing the microwire support and incubated at 37° C. overnight in a 5% CO₂ atmosphere. The following day the culture medium was replaced with fresh medium and the vessel was connected to a pumping system for continuous replacement of the medium. Cell growth on the surface of the microwire support was confirmed by direct observation of the cells on the microwire support surface under the microscope.

### Example 8

### Application of Microwire Supports to the Construction of Static Support Bed (SSB) Devices

In order to produce the microwire support based device, the static support bed (SSB) device, the microwire support was arranged in such a way that a number of continuous, parallel microwire support elements were aligned from top to bottom of every functional unit of the device, being a functional unit the length of the device that only contains whole microwire support elements and being a microwire support element every distinct length of microwire support that goes from top to bottom of a functional unit. The inlet of the microwire support device was connected to the feed-containing vessel through silicon tubing and the outlet of the device was connected, also through silicon tubing, to a three-way valve that led either to the product reservoir or to the waste depending on the regulation of the valve.

### Example 9

### Application of Oligonucleotide-Activated Microwire Support to the Purification of Plasmid DNA

Two oligonucleotides, d[GATC(GAA)₁₇GTATACT] (SEQ ID NO:2) and d[GATCAGTATAC(TTC)₁₇] (SEQ ID NO:3) where 5′-phosphorylated and annealed together to form a double stranded DNA affinity sequence. Plasmid pCMS-EGFP/GAA17 was constructed by inserting the DNA affinity sequence SEQ ID NO:2, into the Bgl II restriction site of PCMS-EGFP. Oligonucleotide-activated micro-wire support was equilibrated for 30 minutes in Binding buffer (2 M NaCl, 0.2 M Sodium acetate, pH 4.5). Then the oligonucleotide-activated micro-wire support was incubated for two hours in Binding buffer containing 10 μg mL−1 plasmid pCMS-EGFP/GAA17 at room temperature. Plasmid pCMS-EGFP/GAA17 contains a (GAA)₁₇ nucleotide sequence (SEQ ID No.:4) that binds to the (CTT)₇ oligonucleotide sequence (SEQ ID NO:1) on the oligonucleotide-activated microwire support. The support was then washed in Binding buffer and a sample was withdrawn for microscopy analysis. The support was then incubated for 1 hour in Elution buffer (1 M tris(hydroxymethyl)aminomethane; 0.05 M ethylenediamine tetraacetic acid, pH 9.5). The material recovered in the Elution buffer contained 1.2 μg mL⁻¹ of plasmid pCMS-EGFP/GAA17 as determined by spectrofluorometry. Plasmid PCMS-EGFP, which is devoid of the complementary nucleotide sequence (GAA)₁₇, was used as negative control.

### Analysis of Plasmid-Loaded Micro-Wire Support:

Oligonucleotide-activated micro-wire support loaded with plasmid pCMS-EGFP/GAA17 as described above was washed in Visualisation buffer (0.1 M NaCl, 0.02 M Sodium acetate, pH 4.5). Then the support was incubated for 10 minutes in a 1:400 dilution of PicoGreen (Molecular Probes; USA), a DNA-binding fluorescent reagent, in Visualisation buffer. The support was then washed in Visualisation buffer and observed in a microscope under fluorescent light. Only oligonucleotide-activated micro-wire support treated with pCMS-EGFP/GAA17 plasmid showed fluorescence due to the emission of light from DNA-binding fluorescent reagent bound to plasmid pCMS-EGFP/GAA17 on the surface of the support.

### Example 10

### Application of SSB Adsorption to the Modification of Immobilized Plasmid DNA Molecules

A microwire support activated with d(CTT)₇ oligonucleotide and BsrB I restriction enzyme was arranged to form a static support bed (SSB) device and this SSB device was connected to vessels using silicon tubing, a pump and a valve as described in previous examples. Binding buffer (2 M NaCl, 0.2 M Sodium acetate, pH 4.5) containing 10 μg mL⁻¹ plasmid pCMS-EGFP/GAA17, which contains two target sites for BsrB I enzyme and a DNA sequence complementary to (CTT)₇, was pumped through the system at room temperature for two hours at 1 cm min⁻¹. Then the feed of the system was changed to Restriction buffer (0.1 M NaCl, 0.02 M Sodium acetate, 10 mM MgCl2, pH 5.5) for 2 hours. Then the feed of the system was changed to Elution buffer (1 M tris(hydroxymethyl)aminomethane; 0.05 M ethylenediamine tetraacetic acid, pH 9.5) while magnetic shaking was applied as described in following examples. The effect of the activated-SSB on the plasmid molecules was assessed by measuring the size of the resulting DNA fragments in an agarose gel following treatment of the modified molecules of plasmid pCMS-EGFP/GAA17 with EcoR I.

### Example 11

### Particulate Material Separation on SSB Devices

The system can be used to separate particles from the fluid in which they are contained as described in the following example:

A SSB device based on microwire support was built as described above. This device was set in a system as described in previous examples. A suspension of magnetic particles in water was continuously pumped at a flow of 1 mL min−1 through the SSB device while an external magnetic field was applied using fixed magnets. When the magnetic particles are settled on the surface of the microwire support, the magnetic particles can be detected through their magnetic interaction with the microwire support. When the concentration of magnetic particles breaking through the SSB device was the same as that of the feeding suspension as measured by turbidimetry, the inlet flow was changed to water and the external magnetic field was eliminated by withdrawing the magnets. The magnetic particles were recovered in the product reservoir of the SSB system.

### Example 12

### Magnetic Shaking of the Static Support Bed

A magnetic shaking procedure has been devised to aid during the elution or de-sorption step from the microwire support during a static support bed process. This is exemplified in the following description: an oligonucleotide-activated static support bed was arranged as described for the production of microwire support devices. A 10 μg mL⁻¹ plasmid pCMS-EGFP/GAA17 solution in Binding buffer (2 M NaCl, 0.2 M Sodium acetate, pH 4.5) was recirculated through the system by pumping at 1 mL min⁻¹ for two hours. Then the flow was changed to Washing binding buffer (0.1 M NaCl, 0.02 M Sodium acetate, pH 4.5) for 5 minutes. Then a shaking movement was applied to the SSB by applying an oscillating external magnetic field while the inlet flow was changed to Elution buffer (1 M tris(hydroxymethyl)aminomethane; 0.05 M ethylenediamine tetraacetic acid, pH 9.5). The material recovered in the eluate contained 2.1 μg mL⁻¹ plasmid pCMS-EGFP/GAA17 as measured by spectrofluorometry.

### Example 13

### Solid Phase Synthesis of DNA on SSB Using Temperature Shifts Induced by Applying Electric Current Through the Microwire Support

Primer-activated microwire support was produced as described in previous examples for oligonucleotide-activated microwire support with the only difference of the substitution of H₂N-d(TTTGTGATGCTCGTCAGGG) oligonucleotide (SEQ ID NO:5) for H₂N-d(CTT)₇ oligonucleotide (SEQ ID NO:1). This primer-activated microwire support was used to produce a static support bed (SSB) as described in previous examples. The metallic core of all the microwire support elements in one end of the static support bed were connected to the positive pole of a power supply, while the metallic core of all the microwire support elements in the other end of the static support bed were connected to the negative pole of the power supply. This SSB was arranged in such a way as to produce a SSB device as described in previous examples where the electric connections of either end of the bed were electrically isolated from the inner space of the SSB device. By applying different electric current between the poles, the temperature of the SSB could be regulated between 30° C. and 95° C. Two thermostatic devices were connected to the ends of the SSB device, in such a way that the temperature of the inflowing and outflowing liquid could be adjusted between 30° C. and 95° C. These devices consisted of water filled coils surrounding the outlet tubing connected to the SSB device. An aqueous solution at pH 8.8 containing 200 μM dNTP, 20 mM Tris-HCl, 10 mM KCl, 10 mM (NH₄)₂SO₄, 0.1% Triton X-100, 0.3 unit mL⁻¹ Taq DNA polymerase, 0.5 μM d(TTTGTGATGCTCGTCAGGG) (SEQ ID NO:5) and 1 μg mL⁻¹ of a linear double stranded DNA fragment containing the sequence TTTGTGATGCTCGTCAGGGAATTC (SEQ ID NO:6) on the 5′ end and the sequence GAATTCCCTGACGAGCATCACAAA (SEQ ID NO:7) on the 3′ end, was continuously recirculated through the system described above while 30 temperature shift cycles, each consisting of 2 minutes at 90° C., 2 minutes at 55° C. and 5 minutes at 72° C., were applied. Then 1 unit mL⁻¹ EcoR I enzyme was added to the solution contained on the system and the temperature was kept at 37° C. for 1 hour. Finally the solution contained in the system was recovered and applied to a gel filtration chromatography system to separate the amplified double stranded DNA fragment from the enzymes and residual reaction components.

### Example 14

### Application of Microwire Supports to the Construction of a Static Support Bed (SSB) Device for Cell Growth

Thirteen bundles of microwire supports were produced in the following way: microwire supports 11 cm in length were arranged in 13 groups of 100 parallel microwire supports per group. A bundle was produced from each of the groups of microwire supports by binding together the ends on one side of the microwire supports and applying melted plastic material. Then the same procedure was applied at the other end of the microwire supports.

Eight filter membranes with 0.2 μm pore size, 10.75 cm long and the shape of hollow fibers with a lumen of 0.5 mm were prepared so that the lumen at one of the ends of every fiber was blocked by collapsing the fiber at that end while the other end remained open.

Then the bundles and the filter membranes were aligned parallel to each other so that four filter membranes had the open end on one side of the alignment and the other four had the open end on the other side. Then all the ends on one side of the alignment were embedded in a plastic disc 15 mm in diameter and 5 mm thick. The ends on the other side of the alignment were embedded in a disc similar to the previous one but perforated in the centre to produce an inoculation port consisting of a 1 mm hole through the disc. The ends of the bundles went though the discs exactly to the point that the surface at the other side of the disc was reached. Open ends of filter membranes went through the discs exactly to the point were the open end was available from the other side of the disc. Closed ends of filter membranes went through the disc half the distance between both sides of the disc.

This arrangement containing the bundles, filter membranes and discs was sterilised and the hole on the disc was sealed with a removable seal. The arrangement was then introduced in sterility conditions in a sterile 11 cm long glass cylinder with a 15 mm internal diameter. Then the discs and the openings of the glass cylinder were sealed together to form the Cell growth SSB device.

The end of the Cell growth SSB device with the non-perforated disc was connected under sterile conditions to silicon tubing and culture media at 37° C. and saturated in a 5% CO₂ atmosphere was fed upwards through the tube until the Cell growth SSB device was partially filled. Then a suspension of a eukaryotic cell line at 1.5×10⁶ cells per mL was injected into the Cell growth SSB device through the inoculation port and the inoculation port was sealed. Silicone tubing was connected to the open side of the Cell growth SSB device and culture media at 37° C. and saturated in a 5% CO₂ atmosphere was continuously fed to the Cell growth SSB device.

1. A static support bed for purification, separation, modification, or immobilization of target chemical entities or target biological entities present in a fluid, wherein said static support bed comprises one or more microwire supports secured by at least one of their respective ends, said microwire supports having a multilayer structure segregated into a central core and one or more coating layers, and being suitable for the attachment of target chemical entities or target biological entities,
wherein the surface of one or more of the microwire supports is modified by one or both of:
a) attachment of one or more ligands directly or through a linker; or
b) by coating it with a functional coating;
wherein such purification, separation, modification, and/or immobilization occurs through attachment of the target chemical entity or target biological entity to the functional coating or to one or more of the one or more ligands present on the surface of the microwire supports; and
wherein the static support bed is placed into the inner volume of a channel;
wherein when a magnetic field acts upon said static support bed then the magnetic field is not used to separate or detect magnetically susceptible particles present in the fluid through the magnetic interaction established between the microwire supports and said magnetically susceptible particles.

2. (canceled)

3. The static support bed according to claim 1, wherein the central core and the coating layers are made of different materials selected from the group consisting of glass, metallic, ceramic, polymeric and plastic material.

4. The static support bed according to claim 3, wherein the central core of at least one of said one or more microwire supports is made of metal, and at least one coating layer of said microwire supports is made of glass.

5. The static support bed according to claim 1, wherein said static support bed has a maximum cross-sectional dimension in the range of about 0.5 cm to about 1.5 m, and a length in the range of about 0.5 cm to about 10 m.

6. The static support bed according to claim 5, wherein the maximum cross-sectional dimension is in the range of about 0.5 cm to about 50 cm and the length is in the range of about 5 cm to about 1.5 m.

7. The static support bed according to claim 1, wherein the microwire supports have a maximum cross-sectional dimension in the range of about 1 μm to about 1000 μm and a length to maximum cross-sectional dimension ratio larger than about 5.

8. The static support bed according to claim 7, wherein the microwire supports have a maximum cross-sectional dimension in the range of about 1 μm to about 100 μm and a length to maximum cross-sectional dimension ratio is one of larger than about 50, larger than about 500, and larger than about 1000.

9. (canceled)

10. (canceled)

11. The static support bed according to claim 1, wherein any given individual microwire support forms an angle that is one or both of between about 0° and about 45° and between about 0° and about 10° with any other neighbouring microwire support.

12. (canceled)

13. (canceled)

14. The static support bed according to claim 1, wherein the surface of the microwire supports is modified by coating the surface with a functional coating selected from the group consisting of polymeric, proteic, gelatin, or collagen coating.

15. (canceled)

16. The static support bed according to claim 1, wherein at least one of the one or more ligands is selected from the group consisting of cells, biological tissues, antibodies, antibiotics, antigens, nucleic acids, peptides, hormones, coenzymes, biological catalysts, chemical catalysts, chemical reactants, lipids, sugars, aminoacids, proteins, nucleotides, a compound containing a functional group selected from the group consisting of diethylaminoethyl, quaternary aminoethyl, quaternary ammonium, carboxymethyl, sulphopropyl, methyl sulphonate, butyl, octal, and phenyl, or mixtures of any thereof.

17. (canceled)

18. The static support bed according to claim 1, wherein the linker is selected from the group consisting of polymeric coating, proteic coating, gelatin coating, collagen coating, cells, antibodies, antigens, nucleic acids, peptides, coenzymes, lipids, sugars, aminoacids, proteins, nucleotides, cyanuric chloride, quinine, p-mercurybenzoate, phenyl boronic acid, and a compound containing a functional group selected from the group consisting of aldehyde, aromatic amine, nitrene, maleimide, carboxylic acid, isocyanate, diethylaminoethyl, quaternary aminoethyl, quaternary ammonium, carboxymethyl, sulphopropyl, methyl sulphonate, butyl, octal, and phenyl, or mixtures of any thereof.

19. A microwire support for the integration in the static support bed of claim 1, said microwire support having a multilayer structure segregated into a central core and one or more coating layers, wherein the surface of the microwire support is modified by one or both of:
a) attachment of one or more ligands directly or through a linker; or
b) by coating it with a functional coating, wherein the coating is a coating which may interact by covalent or non-covalent coupling with the target entity; and, wherein the functional coating is not a polymeric coating.

20. The microwire support according to claim 19, wherein the central core and the coating layers are made of different materials each selected from the group consisting of glass, metallic, ceramic, polymeric and plastic material.

21. The microwire support according to claim 19, wherein the core of said microwire support is made of metal and at least one coating layer is made of glass.

22. The microwire support according to claim 19, wherein the maximum cross-sectional dimension thereof is in the range of about 1 μm to about 1000 μm and its length to maximum cross-sectional dimension ratio is larger than about 5.

23. The microwire support according to claim 19, wherein the surface thereof is modified by coating the surface with a polymeric, proteic, gelatin, or collagen coating.

24. (canceled)

25. A method for purification, separation, modification, and/or immobilization of target chemical entities or target biological entities present in a fluid, using the static support bed defined in claim 1, said method comprising:
a) loading a fluid containing the target chemical entities or target biological entities into the inner volume of a channel containing the static support bed;
b) attaching the target chemical entities or target biological entities to the microwire supports of the static support bed;
c) optionally, carrying out a chemical or biological modification on the target entity;
d) optionally, washing the channel and discharging undesired components and impurities of the fluid;
and
e) eluting the resulting chemical entities or the resulting biological entities;
wherein when a magnetic field acts upon said static support bed then the magnetic field is not used to separate or detect magnetically susceptible particles present in the fluid through the magnetic interaction established between the microwire supports and said magnetically susceptible particles.

26. (canceled)

27. (canceled)

28. The method according to claim 25, for immobilization and cultivation of cells.

29. (canceled)

30. The method according to claim 28, wherein step a) comprises loading a culture media and a suspension of at least one cell line, and optionally continuously loading a culture media into the inner volume of the channel containing the static support bed.

31. The method according to claim 25, further including one or both of:
applying a magnetic field to the static support bed for shaking and/or heating the system; and,
applying an electric current to the static support bed.
